(19) United States
(12) Patent Application Publication
(10) Pub. No.: US 2025/0204765 A1
(43) Pub. Date: Jun. 26, 2025

(54) CIRCUIT DEVICE, IMAGE PICKUP APPARATUS, ENDOSCOPE, AND MANUFACTURING METHOD FOR IMAGE PICKUP APPARATUS

(71) Applicant: OLYMPUS MEDICAL SYSTEMS CORP., Tokyo (JP)

(72) Inventors: Keiichi KOBAYASHI, Tokyo (JP); Junya YAMADA, Tokyo (JP)

(73) Assignee: OLYMPUS MEDICAL SYSTEMS CORP., Tokyo (JP)

(21) Appl. No.: 19/073,671

(22) Filed: Mar. 7, 2025

Related U.S. Application Data
(63) Continuation of application No. PCT/JP2022/033915, filed on Sep. 9, 2022.

(51) Int. Cl. A61B 1/05; A61B 1/00; H04N 23/50

(52) U.S. Cl. CPC A61B 1/05; A61B 1/0011; H04N 23/555

(57) ABSTRACT

A circuit device configured for use in an image pickup apparatus includes a plurality of first pads arranged in a two-dimensional pattern; a plurality of first conductors arranged in a two-dimensional pattern, each first conductor having a first end and a second end, the first ends being connected to the plurality of first pads, respectively; a plurality of lands connected to the second ends of the plurality of first conductors, respectively; a second pad disposed at a central region within the two-dimensional pattern of the plurality of first pads; a second conductor having a third end and a fourth end, the third end being connected to the second pad, the fourth end being connected to a specific pad among the plurality of first pads; and a resist portion covering at least a part of the second conductor but not any of the plurality of first conductors.

### CROSS-REFERENCE TO RELATED APPLICATION

This application is a continuation application of PCT/JP2022/033915 filed on Sep. 9, 2022, the entire contents of which are incorporated herein by this reference.

### BACKGROUND OF THE INVENTION

### 1. Field of the Invention

The present disclosure relates to a circuit device, an image pickup apparatus, an endoscope, an manufacturing method for an image pickup apparatus.

### 2. Description of the Related Art

In recent years, three-dimensional circuit devices, for example, molded interconnect devices (MID) have been used for miniaturization and high functionality of electronic devices.

Japanese Patent Application Laid-Open Publication No. 2017-23234 discloses a camera unit for an endoscope. The camera unit is configured by using an irregularly-shaped circuit substrate which is a three-dimensional circuit device. The camera unit includes an image pickup device, a flat wiring board (flat-plate wiring board) on which electronic components are mounted, and the irregularly-shaped circuit substrate (three-dimensional wiring board). A plurality of cables are bonded to each of a plurality of side surfaces of the irregularly-shaped circuit substrate.

In order to solder-bond electronic components to pads of conductor patterns, a resist pattern, which is formed of solder resists configured to cover around the pads, is placed for preventing short circuit between the pads and preventing solder from flowing out.

WO Publication No. 2009/090896 discloses an electronic device in which an elongated insulating film (solder resist pattern) is placed on a conductor pattern formed on a principal surface of a flat wiring board.

A resist pattern is placed by patterning a spin-coated resist or a film resist using a photolithography method. The resist pattern is sometimes placed by screen-printing of the resist.

### SUMMARY OF THE INVENTION

A circuit device configured for use in an image pickup apparatus according to one aspect of the present disclosure includes: a plurality of first pads arranged in a two-dimensional pattern; a plurality of first conductors arranged in a two-dimensional pattern, each first conductor having a first end and a second end, the first ends being connected to the plurality of first pads, respectively; a plurality of lands connected to the second ends of the plurality of first conductors, respectively; a second pad disposed at a central region within the two-dimensional pattern of the plurality of first pads; a second conductor having a third end and a fourth end, the third end being connected to the second pad, the fourth end being connected to a specific pad among the plurality of first pads; and a resist portion covering at least a part of the second conductor but not any of the plurality of first conductors.

An image pickup apparatus according to one aspect of the present disclosure includes a circuit device; a camera unit comprising a plurality of electrodes including: a first electrode; and a plurality of second electrodes arranged around the first electrode; and wherein the plurality of first pads are soldered to the plurality of second electrodes of the camera unit, respectively, and the second pad is soldered to the first electrode of the camera unit. The circuit device includes: a plurality of first pads arranged in a two-dimensional pattern; a plurality of first conductors arranged in a two-dimensional pattern, each first conductor having a first end and a second end, the first ends being connected to the plurality of first pads, respectively; a plurality of lands connected to the second ends of the plurality of first conductors, respectively; a second pad disposed at a central region within the two-dimensional pattern of the plurality of first pads; a second conductor having a third end and a fourth end, the third end being connected to the second pad, the fourth end being connected to a specific pad among the plurality of first pads; and a resist portion covering at least a part of the second conductor but not any of the plurality of first conductors.

An endoscope according to one aspect of the present disclosure includes: an image pickup apparatus, the image pickup apparatus being disposed in a distal end portion of an insertion portion of the endoscope. The image pickup apparatus includes a circuit device; a camera unit comprising a plurality of electrodes including: a first electrode; and a plurality of second electrodes arranged around the first electrode; and wherein the plurality of first pads are soldered to the plurality of second electrodes of the camera unit, respectively, and the second pad is soldered to the first electrode of the camera unit. The circuit device includes: a plurality of first pads arranged in a two-dimensional pattern; a plurality of first conductors arranged in a two-dimensional pattern, each first conductor having a first end and a second end, the first ends being connected to the plurality of first pads, respectively; a plurality of lands connected to the second ends of the plurality of first conductors, respectively; a second pad disposed at a central region within the two-dimensional pattern of the plurality of first pads; a second conductor having a third end and a fourth end, the third end being connected to the second pad, the fourth end being connected to a specific pad among the plurality of first pads; and a resist portion covering at least a part of the second conductor but not any of the plurality of first conductors.

A manufacturing method for an image pickup apparatus according to one aspect of the present disclosure includes: fabricating a circuit device comprising: a plurality of first pads arranged in a two-dimensional pattern; a plurality of first conductors arranged in a two-dimensional pattern, each first conductor having a first end and a second end, the first ends being connected to the plurality of first pads, respectively; a plurality of lands connected to the second ends of the plurality of first conductors, respectively; a second pad disposed at a central region within the two-dimensional pattern of the plurality of first pads; and a second conductor having a third end and a fourth end, the third end being connected to the second pad, the fourth end being connected to a specific pad among the plurality of first pads; and forming a resist portion using a dispenser, the resist portion being formed to cover at least a part of the second conductor but not any of the plurality of first conductors.

### BRIEF DESCRIPTION OF THE DRAWINGS

FIG. 1 is a perspective view of an image pickup apparatus in a first embodiment.

FIG. 2 is a perspective view of the image pickup apparatus in the first embodiment.

FIG. 3 is a sectional view taken along the line III-III in FIG. 1.

FIG. 4 is a bottom view of a camera unit of the image pickup apparatus in the first embodiment.

FIG. 5 is a bottom view of a cavity of a three-dimensional circuit device in the first embodiment.

FIG. 6 is a bottom view of the cavity of the three-dimensional circuit device in the first embodiment.

FIG. 7 is a bottom view of the cavity of the three-dimensional circuit device in the first embodiment.

FIG. 8 is a flowchart of a manufacturing method of the image pickup apparatus in the first embodiment.

FIG. 9 is a bottom view of a cavity of a three-dimensional circuit device in a first modified example of the first embodiment.

FIG. 10 is a bottom view of a cavity of a three-dimensional circuit device in a second modified example of the first embodiment.

FIG. 11 is a bottom view of a cavity of a three-dimensional circuit device in a third modified example of the first embodiment.

FIG. 12 is a perspective view of an image pickup apparatus in the third modified example of the first embodiment.

FIG. 13 is a perspective view of an endoscope in a second embodiment.

### DETAILED DESCRIPTION OF THE PREFERRED EMBODIMENT(S)

Hereinafter, embodiments of the present disclosure will be described with reference to drawings.

Note that the drawings based on each embodiment are schematic. The relationship between thicknesses and widths of respective parts, a ratio of a thickness of a certain part to that of another part, and the like of the respective parts are different from the actual ones. The respective drawings include parts in which the relationships and ratios among the dimensions are different.

### First Embodiment

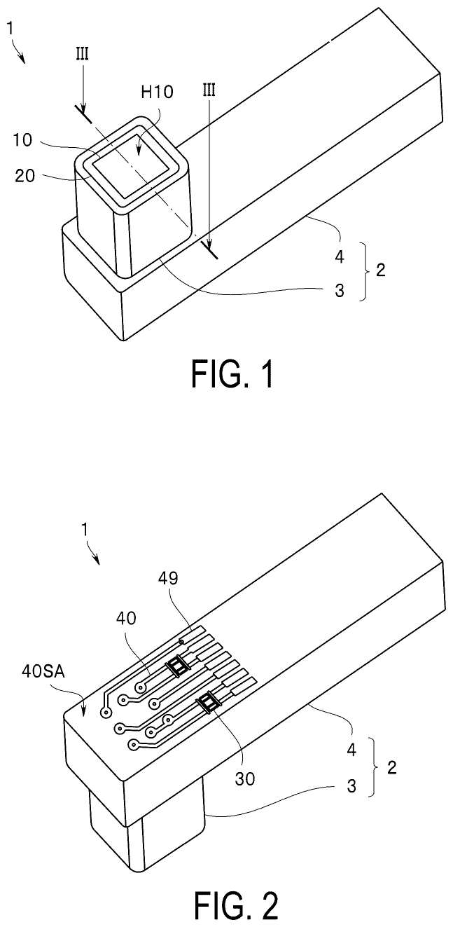

An image pickup apparatus 1 in the present embodiment is shown in FIGS. 1 to 3. The image pickup apparatus 1 includes a three-dimensional circuit device 2 which is an MID (Molded Interconnect Device), and a camera unit 10.

The three-dimensional circuit device 2 is a three-dimensional molded interconnect device formed by placing conductor patterns on the surface of an injection-molded three-dimensional article. Unlike the conventional flat conductor pattern board, using the three-dimensional circuit device 2 enables the shape thereof to have a function, and enables the conductor patterns to be formed on an inclined surface, a vertical surface, a curved surface, and in a through hole, etc.

The three-dimensional circuit device 2 has a complicated three-dimensional structure. Specifically, the three-dimensional circuit device 2 includes a protruded region 3 and an assembling region 4. The protruded region 3 configures a cavity H10 which is a hole in which the camera unit 10 is housed.

A plurality of first pads 61 are placed on a bottom surface H10SB of the cavity H10. The three-dimensional circuit device 2 includes, under the first pads 61, through electrodes 80 which reach a surface 40SA opposite to the bottom surface H10SB. On the surface 40SA, conductor patterns 40 connected to the through electrodes 80 are placed, and electronic components, for example, chip capacitors 30 are surface-mounted to the conductor patterns 40. Although not shown, a signal cable is bonded to a pad 49 of each of the conductor patterns 40.

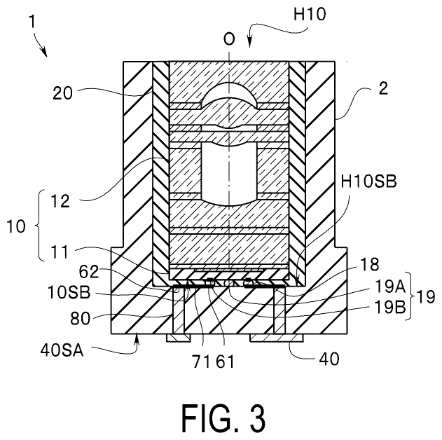

As shown in FIG. 3, a gap between the camera unit 10 housed in the cavity H10 and a wall surface of the cavity H10 is filled with a sealing resin 20. The camera unit 10 includes an imager 11 and an optical unit 12. The optical unit 12 includes a plurality of lenses, and the like. The imager 11 is a CCD, a CMOS, or the like that converts an object image, which is condensed by the optical unit 12, into an electric signal.

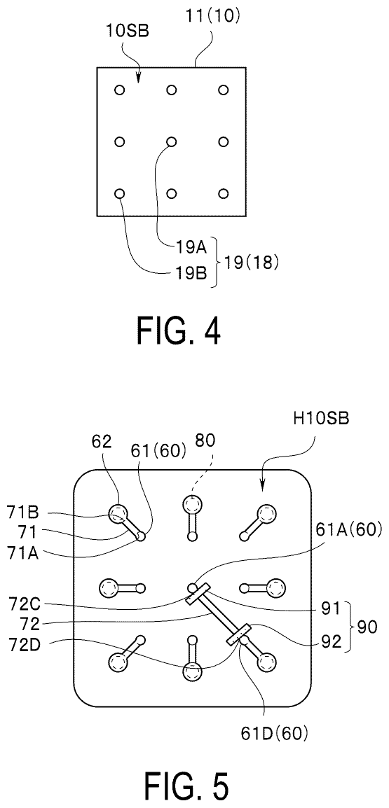

As shown in FIG. 4, the camera unit 10 (the imager 11) includes, on the bottom surface 10SB, a plurality of external electrodes 19 that transmit and receive the electric signal. The external electrodes 19 include a center electrode 19A and eight peripheral electrodes 19B surrounding the center electrode 19A. The plurality of external electrodes 19, which are arranged in a grid shape, each include a solder bump 18, to configure a ball grid array.

As shown in FIG. 5, the three-dimensional circuit device 2 includes, on the bottom surface H10SB, pads 60 bonded to the external electrodes 19 of the camera unit 10 with the solder bumps 18. The pads 60 include the first pads 61 bonded to the peripheral electrodes 19B and a second pad 61A bonded to the center electrode 19A.

First conductor patterns 71 each include a first end 71A and a second end 71B, and the first pads 61 are connected respectively to the first ends 71A of the first conductor patterns 71. The plurality of elongated first conductor patterns 71 are arranged radially outward, with the second pad 61A as a center. The second ends 71B of the first conductor patterns 71 are connected respectively to lands 62 placed on the through electrodes 80. The distance between two adjacent ones of the plurality of lands 62 is longer than the distance between two adjacent ones of the plurality of first pads 61. With such a configuration, a crosstalk of the electric signals is suppressed in the image pickup apparatus 1.

In addition, the second pad 61A, which is bonded to the center electrode 19A of the external electrodes 19 of the camera unit 10, is placed on the bottom surface H10SB. A second conductor pattern 72 includes a third end 72C and a fourth end 73D, and the second pad 61A is connected to the third end 72C of the second conductor pattern 72. The fourth end 72D of the second conductor pattern 72 is connected to a dummy pad 61D of the plurality of first pads 61.

The dummy pad 61D is a conductor pattern for arranging the plurality of first pads 61 at four-fold symmetric positions. The dummy pad 61D does not have to be connected to the through electrode 80. In addition, the peripheral electrode 19B connected to the through electrode 80 to which the dummy pad 61D is connected does not have to be connected to an internal circuit of the camera unit 10. The dummy pad 61D may be equipotential with the second pad 61A, for example, a ground potential.

The three-dimensional circuit device 2 includes, on the bottom surface H10SB, a resist pattern 90 configured to cover at least a part of the second conductor pattern 72 and not to cover the plurality of first conductor patterns 71. The resist pattern 90 includes a first resist pattern 91 and a second resist pattern 92, each of which intersects with the second conductor pattern 72. The first resist pattern 91 covers the third end 72C of the second conductor pattern 72, and the second resist pattern 92 covers the fourth end 72D of the second conductor pattern 72.

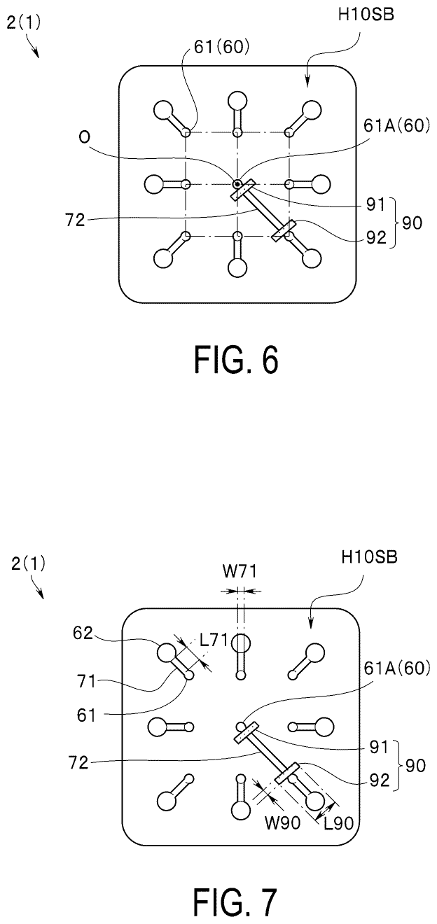

As shown in FIG. 6, the plurality of first pads 61 are arranged at the four-fold symmetric positions, with the second pad 61A, which is located at the optical axis position, as a center O. In other words, the plurality of first pads 61 are arranged at positions such that one is superimposed on another when the plurality of first pads 61 are rotated around the center O by 90 degrees. Similarly, the plurality of first conductor patterns 71 are arranged at the four-fold symmetric positions, with the second pad 61A as a center O.

As shown in FIG. 7, the plurality of first conductor patterns 71 has the same lengths L71, and substantially the same widths W71.

The bottom surface 10SB of the camera unit 10 shown in FIG. 4 is not fixed to the bottom surface 10SB of the cavity H10 before the reflow soldering step. The camera unit 10, which is not fixed, moves in a direction in which the solder flows out, when the solder bumps 18 of the external electrodes 19 melt, to thereby cause connecting members to move in the direction in which the solder flows out, due to the surface tension of the solder. In view of the above, the plurality of pads 60 may be surrounded by the solder resist, to thereby suppress the flowing out of the solder. However, there is a difficulty in placing the resist patterns to cover the peripheries of the plurality of pads 60 on the small surface of the three-dimensional circuit device 2.

It is not easy to place the resist patterns on the surface of the three-dimensional circuit device. In other words, unlike the flat wiring board, it is difficult to apply spin coating or screen printing to the surface of the three-dimensional circuit device. In addition, it is also difficult to pattern the resist located on the surface of the three-dimensional circuit device using the photolithography method.

When the camera unit is solder-bonded to the pads of the three-dimensional circuit device, if the solder resist patterns covering the peripheries of the pads are not placed, there has been a possibility that the solder flows out to the conductor patterns around the pads, to thereby cause the camera unit to move from a predetermined position. If the optical axis of the camera unit shifts from the predetermined position, a desired field of view cannot be obtained, which has led to a possibility that the performance of the image pickup apparatus degrades.

However, in the image pickup apparatus 1, the pads 60 are respectively connected only to the first conductor patterns 71, to thereby allow the solders 18 to flow out to the first conductor patterns 71. When the solder 18 of the peripheral electrode 19B bonded to the dummy pad 61D melts, the solder 18 is allowed to flow only to the first conductor pattern 71 due to the presence of the second resist pattern 92. In addition, when the solder 18 of the center electrode 19A bonded to the second pad 61A melts, the solder 18 is prevented from flowing out to the periphery due to the presence of the first resist pattern 91.

As already described above, the plurality of first conductor patterns 71 are arranged at the four-fold symmetric positions, with the second pad 61A as the center O. With such a configuration, when the plurality of solders 18 that are arranged symmetrically with respect to the center O melt, the solders 18 flow out to the plurality of first conductor patterns 71 arranged symmetrically with respect to the center O. The plurality of melted solders 18 move symmetrically with respect to the center O, to thereby cause the image pickup apparatus 1 to be pulled due to the surface tension of the solders 18. In the image pickup apparatus 1, the peripheral electrodes 19B are arranged at the positions of the first pads 61 and the center electrode 19A is arranged at the position of the second pad 61A, due to what is called a self-alignment effect.

Note that the plurality of first pads 61 and the plurality of first conductor patterns 71 may be arranged at the N-fold symmetric positions (N is a natural number equal to or greater than 2), with the second pad 61A as the center O.

In the present embodiment, the camera unit 10 is housed in the cavity H10 of the three-dimensional circuit device 2. It is not easy to place the resist pattern for solder-bonding the external electrodes located on a lower surface of the camera unit housed in the cavity of the three-dimensional circuit device and the pads located on the bottom surface of the cavity. It is difficult to fix the small lower surface of the camera unit at the predetermined position on the bottom surface of the cavity before the reflow soldering step. Therefore, there is a case where the solders melt and the solders flow out to the conductor patterns around the pads in the reflow soldering step, to thereby sometimes cause the camera unit to move from the predetermined position. If the optical axis of the camera unit shifts from the predetermined position, a desired field of view cannot be obtained, which results in a possibility that the characteristics of the image pickup apparatus deteriorate.

In particular, the image pickup apparatus 1 is small, and the area of the bottom surface H10SB of the cavity H10 is 4 mm², for example, that is less than 5 mm². Furthermore, for the purpose of miniaturization of the image pickup apparatus 1, the cavity H10 is formed such that the wall surfaces of the cavity are substantially perpendicular to the bottom surface H10SB. There is a case where the wall surfaces of the cavity H10 incline, but the inclination angle of the wall surfaces are only few degrees. Therefore, it is further difficult to place the resist pattern on the bottom surface H10SB.

However, in the three-dimensional circuit device 2, no position shift (optical axis position shift and rotation) occurs in the camera unit 10 housed in the cavity H10 when the solders melt (reflow soldering step). The image pickup apparatus 1 achieves a high performance, since a desired field of view can be obtained without the position shift of the center (optical axis) O of the camera unit 10.

Note that the plurality of first pads 61 and the plurality of first conductor patterns 71 may be arranged at the N-fold symmetric positions (N is a natural number equal to or greater than 2), with the second pad 61A as the center O.

The plurality of first conductor patterns 71 to which the solders flow out may have substantially the same length L71 and width W71, in order to prevent the position shift of the camera unit 10. Note that the expression “substantially the same” indicates, for example, the state where the respective lengths of the first conductor patterns 71 is more than 85% and less than 115% of an average value of the lengths of the plurality of first conductor patterns 71.

As described later, the resist patterns 90 are placed by using a dispenser. Therefore, each of the resist patterns 90 has an elongated shape. For example, as shown in FIG. 7, the length L90 of the first resist pattern 91 is more than three times the width W90 thereof.

Note that, when the image pickup apparatus 1 is in the state of a finished product, it is impossible to directly identify that the resist patterns 90 have been placed by using the dispenser.

The resist patterns 90 can be easily placed even on the bottom surface H10SB on which it is difficult to place a resist pattern covering the peripheries of all the pads 60. Furthermore, compared with the resist pattern covering the peripheries of all the pads 60, the amount of resist to be used in the resist patterns 90 is small, which results in a reduction in the cost of the resist patterns 90 and a reduction in the take time for placing the resist.

### Manufacturing Method of Image Pickup Apparatus

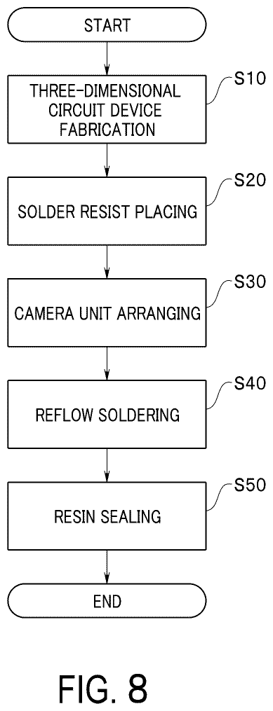

The manufacturing method of the image pickup apparatus 1 will be described with reference to the flowchart in FIG. 8.

### <Step S10>Three-Dimensional Circuit Device Fabrication Step

A molded body is fabricated by injection molding. In other words, an MID resin is injected into a mold (not shown) having the shape of the three-dimensional circuit device 2. The surface of the molded body is irradiated with laser, to thereby form a region having a catalytic activity of electroless plating. Furthermore, through holes are formed in the bottom surface H10SB of the cavity H10, and the conductors are put in the through holes. After that, electroless plating processing is performed, and the molded body becomes the three-dimensional circuit device (three-dimensional circuit device 2) in which the conductors are placed on the surface thereof. Each of the conductors includes, on the copper layer thereof, a barrier layer formed of nickel/gold, for example.

In other words, the three-dimensional circuit device includes the following components placed on the bottom surface of the cavity in which the camera unit is housed. The components include: a plurality of first pads arranged at the four-fold symmetric positions, the plurality of first pads being solder-bonded respectively to a plurality of peripheral electrodes of a plurality of external electrodes of the camera unit; a plurality of first conductor patterns arranged at the four-fold symmetric positions, the plurality of first conductor patterns each including the first end and the second end, the first ends of the plurality of first conductor patterns being connected respectively to the plurality of first pads; a plurality of lands connected respectively to the second ends of the plurality of first conductor patterns; a second pad arranged at the center of symmetry of the plurality of first pads, the second pad being solder-bonded to the center electrode of the plurality of external electrodes of the camera unit; and a second conductor pattern including the third end and the fourth end, the third end being connected to the second pad, the fourth end being connected to a dummy pad of the plurality of first pads.

<Step S20>Solder Resist Placing Step By using the dispenser, the elongated resist patterns 90 are placed on the bottom surface H10SB of the cavity H10.

In other words, the resist patterns 90, which cover at least a part of the second conductor pattern 72 but which do not cover the plurality of first conductor patterns 71, are placed on the bottom surface H10SB by using the dispenser.

### <Step S30>Camera Unit Arranging Step

For example, the camera unit 10, which is a wafer level camera, is fabricated by cutting a stacked wafer formed by adhering image pickup device chips on a plurality of optical wafers. The solder bumps 18 are placed on the external electrodes 19 of the camera unit 10. Instead of the solder bumps 18, solder paste may be placed on the external electrodes 19.

Needless to say, the camera unit 10 may be fabricated before the fabrication of the three-dimensional circuit device (S10) and the placing of the solder resist (S20).

Then, the camera unit 10, in which each of the plurality of external electrodes 19 has the solder bump 18, is housed in the cavity H10 of the three-dimensional circuit device 2. The camera unit 10 is arranged such that the external electrodes 19 (solder bumps 18) abut against the pads 60 on the bottom surface H10SB.

### <Step S40>Reflow Soldering Step

For example, the three-dimensional circuit device 2 is heated to a temperature at which the solder bumps 18 melt by using the reflow furnace. The solders 18 spread to the first conductor patterns 71. When the temperature of the three-dimensional circuit device 2 returns to the room temperature, the external electrodes 19 of the camera unit 10 are solder-bonded to the pads 60 of the three-dimensional circuit device 2.

### <Step S50>Resin Sealing Step

After the reflow soldering step, the sealing resin 20 is injected into the gap between the cavity and the camera unit to be cured, to thereby complete the image pickup apparatus 1.

It is not easy to place the resist patterns surrounding the pads on the bottom surface H10SB of the cavity H10 of the three-dimensional circuit device 2. However, the line-shaped resist patterns 90 are placed only on the second conductor pattern 72 by using the dispenser, the directions in which the plurality of solders 18 flow out to the first conductor patterns 71 are symmetric with the center electrode 19A as a center. With such a configuration, in the image pickup apparatus 1, no position shift occurs in the camera unit 10 housed in the cavity H10. The present manufacturing method enables the image pickup apparatus having a high performance to be manufactured.

### Modified Examples of First Embodiment

Image pickup apparatuses 1A to 1D in the modified examples of the first embodiment are similar to and have the same effects as the image pickup apparatus 1 in the first embodiment. Therefore, in the description below, the same constituent elements having the same functions as those of the image pickup apparatus 1 are attached with the same reference signs and descriptions thereof will be omitted.

### Modified Example 1

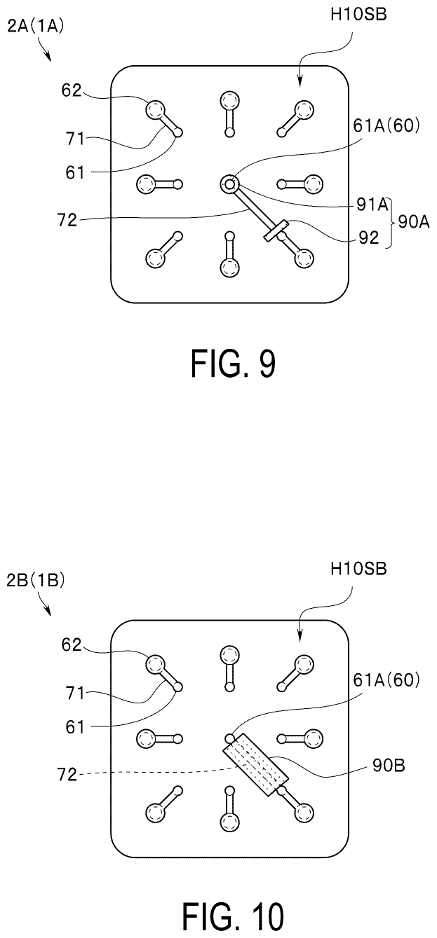

As shown in FIG. 9, in the image pickup apparatus 1A in the present modified example, a first resist pattern 91A of a resist pattern 90A has a ring shape surrounding a second pad 61A.

### Modified Example 2

As shown in FIG. 10, in the image pickup apparatus 1B in the present modified example, a resist pattern 90B covers the entirety of the second conductor pattern 72.

### Modified Example 3

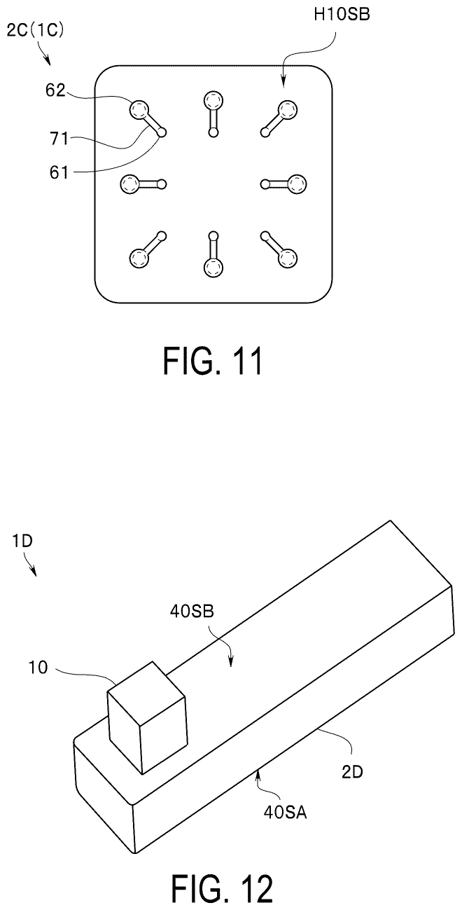

In the image pickup apparatus 1C in the present modified example shown in FIG. 11, the camera unit includes no center electrode. In this configuration, the three-dimensional circuit device 2C includes, on a bottom surface H10SB, a plurality of first pads 61 arranged at four-fold symmetric positions, a plurality of elongated first conductor patterns 71 placed at the four-hold symmetric positions, and a plurality of lands 62 connected respectively to the plurality of first conductor patterns 71.

### Modified Example 4

In the image pickup apparatus 1D in the present modified example shown in FIG. 12, a three-dimensional circuit device 2D includes no cavity. The camera unit 10 is surface-mounted on a surface 40SB of the three-dimensional circuit device 2D. The three-dimensional circuit device 2D includes a through electrode (not shown) formed from the surface 40SB to reach a surface 40SA.

### Second Embodiment

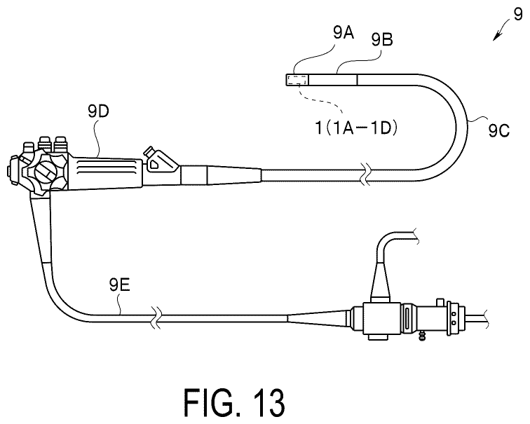

An endoscope 9 in the present embodiment shown in FIG. 13 includes a rigid distal end portion 9A in which the image pickup apparatus 1 (1A to 1D) is provided, a bendable bending portion 9B provided continuously with the proximal end of the rigid distal end portion 9A, and an elongated flexible portion 9C provided continuously with the proximal end of the bending portion 9B. The bending portion 9B is bent by operating an operation portion 9D. The rigid distal end portion 9A, the bending portion 9B, and the flexible portion 9C constitute an insertion portion configured to be inserted into a body. A universal cord 9E extended from the operation portion 9D is connected to a processor, etc., not shown.

The endoscope 9 includes the image pickup apparatus 1 (1A to 1D), and thereby achieves a high performance.

Note that the endoscope 9 is a flexible endoscope for medical use. However, an endoscope in another embodiment may be an endoscope for industrial use, and may be a rigid endoscope having a rigid straight pipe, instead of the flexible portion 9C.

The three-dimensional circuit device is not limited to the MID. The three-dimensional circuit device may be fabricated by processing using a 3D-printer, or cutting processing, for example. The material of the three-dimensional circuit device is not limited to the resin, but ceramics or glass epoxy may be used as the material.

The present disclosure is not limited to the above-described embodiments, and the like, but various changes, modifications, etc., are possible without changing the gist of the present disclosure.
Example 1. An image pickup apparatus comprising a camera unit and a three-dimensional circuit device,
   the three-dimensional circuit device comprising:
   a plurality of first pads arranged at N-fold symmetric positions (N is a natural number equal to or greater than 2), the plurality of first pads being solder-bonded respectively to a plurality of peripheral electrodes of a plurality of external electrodes of the camera unit;
   a plurality of first conductor patterns arranged at the N-fold symmetric positions, the plurality of first conductor patterns each including a first end and a second end, the first ends of the plurality of first conductor patterns being connected respectively to the plurality of first pads;
   a plurality of lands connected respectively to the second ends of the plurality of first conductor patterns;
   a second pad arranged at a center of symmetry of the plurality of first pads, the second pad being solder-bonded to a center electrode of the plurality of external electrodes of the camera unit;
   a second conductor pattern including a third end and a fourth end, the third end being connected to the second pad, the fourth end being connected to a dummy pad of the plurality of first pads; and
   a resist pattern configured to cover at least a part of the second conductor pattern but not to cover the plurality of first conductor patterns.
Example 2. The image pickup apparatus according to Example 1, wherein
   the resist pattern includes a first resist pattern and a second resist pattern that intersect with the second conductor pattern, and the first resist pattern covers the third end and the second resist pattern covers the fourth end.
Example 3. The image pickup apparatus according to Example 2, wherein each of the first resist pattern and the second resist pattern has a length more than three times a width.
Example 4. The image pickup apparatus according to Example 2, wherein the first resist pattern surrounds the second pad.
Example 5. The image pickup apparatus according to Example 1, wherein the resist pattern covers an entirety of the second conductor pattern.
Example 6. An endoscope comprising an image pickup apparatus in a distal end portion of an insertion portion of the endoscope,
   the image pickup apparatus comprising a camera unit and a three-dimensional circuit device,
   the camera unit comprising a plurality of external electrodes including a plurality of peripheral electrodes,
   the three-dimensional circuit device comprising:
      a plurality of first pads arranged at N-fold symmetric positions (N is a natural number equal to or greater than 2), the plurality of first pads being solder-bonded respectively to the plurality of peripheral electrodes;
      a plurality of first conductor patterns placed at the N-fold symmetric positions, the plurality of first conductor patterns each including a first end and a second end, the first ends of the plurality of first conductor patterns being connected respectively to the plurality of first pads;
      a plurality of lands connected respectively to the second ends of the plurality of first conductor patterns;
      a second pad arranged at a center of symmetry of the plurality of first pads, the second pad being solder-bonded to a center electrode of the plurality of external electrodes of the camera unit; and
      a second conductor pattern including a third end and a fourth end, the third end being connected to the second pad, the fourth end being connected to a dummy pad of the plurality of first pads; and
      a resist pattern configured to cover at least a part of the second conductor pattern but not to cover the plurality of first conductor patterns.
Example 7. The endoscope according to Example 6, wherein
   the resist pattern includes a first resist pattern and a second resist pattern that intersect with the second conductor pattern, and the first resist pattern covers the third end and the second resist pattern covers the fourth end.
Example 8. A manufacturing method of an image pickup apparatus, comprising:
   fabricating a three-dimensional circuit device,
   the three-dimensional circuit device comprising:
      a plurality of first pads arranged at N-fold symmetric positions (N is a natural number equal to or greater than 2);
      a plurality of first conductor patterns arranged at the N-fold symmetric positions, the plurality of first conductor patterns each including a first end and a second end, the first ends of the plurality of first conductor patterns being connected respectively to the plurality of first pads;
      a plurality of lands connected respectively to the second ends of the plurality of first conductor patterns;
      a second pad arranged at a center of symmetry of the plurality of first pads; and
      a second conductor pattern including a third end and a fourth end, the third end being connected to the second pad, the fourth end being connected to a dummy pad of the plurality of first pads;
   placing a resist pattern by using a dispenser, the resist pattern being configured to cover at least a part of the second conductor pattern but not to cover the plurality of first conductor patterns;
   arranging a camera unit in the three-dimensional circuit device, the camera unit including a plurality of external electrodes that include a center electrode and a plurality of peripheral electrodes; and
   solder-bonding the plurality of external electrodes respectively to the plurality of first pads, and solder-bonding the center electrode to the second pad.
Example 9. The manufacturing method of the image pickup apparatus according to Example 8, wherein in the solder-bonding, solders spread to the first conductor patterns.
Example 10. The manufacturing method of the image pickup apparatus according to Example 8, wherein
   the resist pattern includes a first resist pattern and a second resist pattern, each of the first resist pattern and the second resist pattern is formed in an elongated shape that intersects with the second conductor pattern, and the first resist pattern is configured to cover the third end and the second resist pattern covers the fourth end.

What is claimed is:

1. A circuit device configured for use in an image pickup apparatus, comprising:
a plurality of first pads arranged in a two-dimensional pattern;
a plurality of first conductors arranged in a two-dimensional pattern, each first conductor having a first end and a second end, the first ends being connected to the plurality of first pads, respectively;
a plurality of lands connected to the second ends of the plurality of first conductors, respectively;
a second pad disposed at a central region within the two-dimensional pattern of the plurality of first pads;
a second conductor having a third end and a fourth end, the third end being connected to the second pad, the fourth end being connected to a specific pad among the plurality of first pads; and
a resist portion covering at least a part of the second conductor but not any of the plurality of first conductors.

2. The circuit device according to claim 1, wherein
the circuit device is a three-dimensional circuit device,
the plurality of first pads are arranged in N-fold symmetry, where N represents a natural number equal to or greater than two,
the plurality of first conductors are arranged in N-fold symmetry,
the second pad is disposed at a center of symmetry of the plurality of the first pads, and
the specific pad is a dummy pad.

3. The circuit device according to claim 1, wherein
the resist portion comprises:
a first resist portion intersecting the second conductor and covering the third end; and
a second resist portion intersecting the second conductor and covering the fourth end.

4. The circuit device according to claim 3, wherein each of the first resist portion and the second resist portion has a length that is more than three times a width thereof.

5. The circuit device according to claim 3, wherein the first resist portion surrounds the second pad.

6. The circuit device according to claim 1, wherein the resist portion covers an entire surface of the second conductor.

7. The circuit device according to claim 1, wherein the second pad is disposed at a center of the two-dimensional pattern of the plurality of first pads.

8. The circuit device according to claim 1, wherein the second conductor is disposed within the two-dimensional pattern of the plurality of first pads.

9. The circuit device according to claim 1, further comprising:
a first surface on which the plurality of first pads, the plurality of first conductors, the plurality of lands, the second pad, the second conductor, and the resist portion are arranged;
a second surface opposite to the first surface; and
at least one through-hole connecting the first surface and the second surface.

10. The circuit device according to claim 9, further comprising:
a plurality of the through-holes; and
a plurality of third conductors provided in the plurality of through-holes and connected to the plurality of lands, respectively.

11. The circuit device according to claim 1, wherein each of the plurality of first conductors extends towards the second pad.

12. The circuit device according to claim 1, wherein the second end of the first conductor is larger than the first end of the first conductor.

13. The circuit device according to claim 1, wherein the second conductor is longer than the first conductor.

14. An image pickup apparatus comprising:
the circuit device according to claim 1;
a camera unit comprising a plurality of electrodes including:
a first electrode; and
a plurality of second electrodes arranged around the first electrode; and
wherein the plurality of first pads are soldered to the plurality of second electrodes of the camera unit, respectively, and
the second pad is soldered to the first electrode of the camera unit.

15. An endoscope comprising the image pickup apparatus according to claim 14, the image pickup apparatus being disposed in a distal end portion of an insertion portion of the endoscope.

16. The endoscope according to claim 15, wherein
the resist portion comprises:
a first resist portion intersecting the second conductor and covering the third end; and
a second resist portion intersecting the second conductor and covering the fourth end.

17. A manufacturing method for an image pickup apparatus, comprising:
fabricating a circuit device comprising:
a plurality of first pads arranged in a two-dimensional pattern;
a plurality of first conductors arranged in a two-dimensional pattern, each first conductor having a first end and a second end, the first ends being connected to the plurality of first pads, respectively;
a plurality of lands connected to the second ends of the plurality of first conductors, respectively;
a second pad disposed at a central region within the two-dimensional pattern of the plurality of first pads; and
a second conductor having a third end and a fourth end, the third end being connected to the second pad, the fourth end being connected to a specific pad among the plurality of first pads; and
forming a resist portion using a dispenser, the resist portion being formed to cover at least a part of the second conductor but not any of the plurality of first conductors.

18. The manufacturing method for the image pickup apparatus according to claim 17, further comprising:
placing a camera unit in the circuit device, the camera unit comprising a plurality of electrodes that include a first electrode and a plurality of second electrodes arranged around the first electrode; and
soldering the plurality of second electrodes to the plurality of first pads, respectively, and soldering the first electrode to the second pad.

19. The manufacturing method for the image pickup apparatus according to claim 18, wherein when the plurality of second electrodes are soldered to the plurality of first pads, respectively, the solder spreads on the plurality of first conductors.

20. The manufacturing method of the image pickup apparatus according to claim 17, wherein the resist portion comprises:
a first resist portion formed in a line shape to intersect the second conductor and cover the third end; and
a second resist portion formed in a line shape to intersect the second conductor and cover the fourth end.
